(19) United States
(12) Patent Application Publication
(10) Pub. No.: US 2016/0324515 A1
(43) Pub. Date: Nov. 10, 2016

(54) SINGLE INCISION SPECIMEN RETRIEVAL ASSEMBLY, METHOD OF USE AND KIT

(71) Applicant: BEACON SURGICAL, INC.LLC, Dobbs Ferry, NY (US)

(72) Inventors: Sundaram Ravikumar, Briarcliff Manor, NY (US); Harry Allan Alward, Shelton, CT (US)

(73) Assignee: Beacon Surgical Instruments, LLC, Dobbs Ferry, NY (US)

(21) Appl. No.: 15/111,595

(22) PCT Filed: Mar. 6, 2015
(86) PCT No.: PCT/US15/19218

§ 371 Date: Jul. 14, 2016

Related U.S. Application Data
(60) Provisional application No. 61/949,806, filed on Mar. 7, 2014.

(51) Int. Cl. A61B 17/00; A61B 17/221; A61B 17/34

(52) U.S. Cl. CPC A61B 17/00234; A61B 17/3417; A61B 17/221; A61B 2017/00349; A61B 2017/00353; A61B 2017/00287; A61B 2017/3454; A61B 2017/2212

(57) ABSTRACT

A specimen retrieval assembly including a housing containing a specimen retrieval bag and pliable spring, a distal end including a non-blunt tip, and a deployment mechanism. The specimen retrieval bag is moved from the stowed position within a hollow housing to a deployed position by sliding a rod from a retracted position partially within the hollow housing to an extended position that projects at least partially out of the hollow housing. Two portions of a flexible support ring unfold under spring bias from a folding position as the rod enters the extended position to open a mouth of the specimen retrieval bag. The distal end of the assembly has a non-blunt tip which is shaped to puncture human skin.

### RELATED APPLICATIONS

This application claims priority from provisional application Ser. No. 61/949,806 filed Mar. 7, 2014 and PCT Application No PCT/US2015/019,218 filed Mar. 6, 2015.

### FIELD OF THE INVENTION

The present invention relates to a specimen retrieval assembly, its method of use, and a kit containing such assembly.

### BACKGROUND OF THE INVENTION

In general, laparoscopic surgery is a minimally invasive surgical technique, in which surgery is performed through several small incisions rather than the traditional larger incision. This technique relies on the use of endoscopes and long-handled instruments that are introduced into the body through an insertion port, or trocar. As endoscope and instrument technology has improved, the technique has become more and more prevalent and has been adapted to virtually every imaginable procedure. Today, laparoscopic surgery is one of the most common surgical techniques in the United States.

The initial opening in the body tissue to allow passage of surgical instruments to the interior of the body may be a natural passageway of the body, or it can be created by a tissue piercing instrument such as a trocar, or created by a small incision into which a cannula, is inserted. Because the housings, instrumentation, and any required punctures or incisions are relatively small in laparoscopic surgery, the surgery is less invasive as compared to conventional surgical procedures in which the surgeon is required to cut open large areas of body tissue. Therefore, minimally invasive surgery or laparoscopic surgery minimizes trauma to the patient and reduces patient recovery time and hospital costs. In addition, in certain surgeries the abdominal walls and abdominal organs are minimally damaged during such a surgical operation, so that complications, recovery time of a patient and pain during the recovery of the patient can all be reduced.

Minimally invasive procedures may be used for partial or total removal of body tissue or organs from the interior of the body, e.g. nephrectomy, cholecystectomy, lobectomy, myomectomy, hysterectomy and other procedures including thoracic, laparoscopic and endoscopic procedures. During such procedures, it is common that a cyst, tumor, or other affected tissue or organ needs to be removed via the access opening in the skin, or through a cannula. Various types of entrapment devices have been disclosed to facilitate this procedure. In many procedures where cancerous tumors are removed, removal of the specimen in an enclosed environment is highly desirable to prevent seeding of cancer cells. Further, in certain types of surgery such as cholecystectomy, an infected or inflamed gallbladder is removed and bile must be contained so as not to contaminate the surgical area.

Many types of laparoscopic surgery involve removal of tissue, and virtually all of these surgeries require the use of a specimen pouch, also called an endoscopic specimen bag or an “endo bag” or a laparoscopic bag or “lap-bag”. The bag is inserted into the body via a cannula and trocar, in an incision location where another device has been removed or for example through the belly button or umbilicus, and the bag is deployed within the body cavity to receive the tissue, closed, and then removed through one of the incisions, typically the umbilicus, or in females the vagina if a gynecological surgery.

During the abdominal surgery using an endoscope as described above, when an operator performs an operation of removing tumors or suturing internal organs and the like, the operator inserts various medical instruments into an abdomen, together with the endoscope and surgical instruments, to take pieces, of tumors or internal organs, which are removed during the abdominal surgery, out of the abdomen by receiving the pieces of tumors or internal organs in a vinyl bag using a medical instrument referred to as a lap-bag.

Conventional lap-bag mechanisms employed in an abdominal surgery using the trocar as described above are well known and comprise a housing or cannula, a wire spring and a folded vinyl bag. In use, the wire spring is deployed from the housing, the bag (including a pull string if the bag will be sealable and pulled out of the body via the string) is deployed, the bag is placed by the surgeon over the specimen, the specimen placed in the open bag by a second instrument via a different incision or trocar, and if included a pull string is engaged sealing the bag holding the specimen, otherwise graspers or other instruments may position a sealing mechanism such as a ring around the opening of the bag to contain the specimen.

In certain surgical operations such as abdominal surgery, an incision is made in the abdomen sufficient for insertion of 10 mm trocar which forms pneumoperitoneum by putting gas (e.g. CO2 gas) into the abdomen during endoscopic surgery so as to create the space and visibility for the endoscopic surgery, and allows an endoscope, a surgical instrument and a medical instrument, such as an endoloop or lap-bag, to be inserted into the abdomen. The trocar and cannula are used for inserting surgical instruments into the abdomen. Given the diameter of the trocar and the various instruments passing therethrough the incision must be at least 15-20 mm.

Conventional minimally invasive surgeries for cholecystectomy involve the use of four trocars (access devices). In general, one trocar is inserted in the umbilicus, through which an endoscope is inserted, with two trocars being inserted on the right side of the abdomen for retraction and mobilizing the gallbladder, in order to identify the important structures. The fourth trocar is typically inserted in the midline above the umbilicus.

The aforementioned method has become the standard approach and has withstood twenty years of changes in surgical skill sets, in various groups of surgeons. However, even using a 5 mm trocar still leaves a scar and thus there is a need to reduce scarring and need for even small trocars such as a 5 mm version. Relatively recently, even newer and advantageous techniques for cholecystectomy have been developed that involve either two 5 mm trocars or even only a single trocar or “port”, called SILS (single incision laparoscopic surgery). As most conventional specimen retrieval bag devices are 10 mm they in turn require a 10 mm trocar opening and thus the less invasive two 5 mm trocar method of surgery cannot be used. Further, the prediction is that nearly twenty to forty percent of all gallbladder surgeries will be performed using SILS as the opening and incision is larger than the two trocar method, but the number of openings or incisions is reduced. This technology involves inserting a single port inserted through the umbilicus, with all the instruments going into the abdominal cavity through the single port. Retrieving the gallbladder is challenging with this technology, especially if the gallbladder is distended due to inflammation.

In conventional cholecystectomy an endoscope or other surgical instruments cannot be inserted through the trocar into which the lap-bag is inserted such that the number of trocars through which an endoscope or surgical instruments can be inserted are increased. This may cause unnecessary surgical damage of the abdomen, increase of the total operation time required for the abdominal surgery and increase possible complications. Other surgeries are also impeded by this approach in that not all instruments may reside within a single trocar, no matter how large the trocar size. Even a 5 mm trocar leaves a scar and may increase complications, pain and recovery time. Thus, a need exists to reduce the size of the main incision during a laparoscopic surgery, typically the incision in which the majority of the instruments will pass through the body wall into the body cavity during the surgery.

Further, a need exists to reduce the number of incisions during surgery and therefore reduce the potential areas for complications, infection and scarring. Specifically a need exists for a specimen retrieval device which does not need a trocar. One less trocar is thus required. A need exists for a self-inserting specimen retrieval device.

This and other needs are met by the inventive specimen removal assembly. The number of incisions, namely larger incisions needed for a 10 mm or 20 mm trocar, are reduced. Further, the total number of incisions are reduced and total number of trocars needed are reduced, even smaller diameter trocars. In addition, an operation process for removing the specimen can be simplified by using the inventive specimen retrieval assembly and the time and cost for the surgery can be reduced.

Accordingly, there remains a need in the art for the inventive specimen retrieval device, its method of use and a kit containing the inventive device. The present invention provides a solution for these needs and other needs.

The present invention has been made to solve the above problems occurring in the prior art and other needs in regard to surgical instruments and methods of treatment.

### SUMMARY OF THE INVENTION

In one aspect, a surgical instrument and assembly for laparoscopic procedures is provided, which is adapted and configured to be self-inserted and retrieve a specimen via an endoscopic bag.

In yet another aspect, a method for laparoscopic procedures is provided including use of a surgical instrument for laparoscopic procedures, which is adapted and configured to be self-inserted and retrieve a specimen via an endoscopic bag.

In another aspect, a kit for laparoscopic procedures is provided including a device which is adapted and configured to be self-inserted and retrieve a specimen via an endoscopic bag.

### DESCRIPTION OF THE DRAWINGS

The above and other advantages of the present invention will become readily apparent with reference to the following detailed description when considered in conjunction with the accompanying drawings which are incorporated in and constitute part of this specification, are included to illustrate and provide a further understanding of the devices and related methods of the invention. Together with the description, the drawings serve to explain the principles of the invention, wherein:

FIG. 1 is a perspective view illustrating one embodiment of the inventive specimen retrieval device with the specimen retrieval bag retracted within the housing.

FIG. 2 is a perspective of a cut-away view illustrating one embodiment of the inventive specimen retrieval device with the specimen retrieval bag retracted within the housing.

FIG. 3 is a perspective view of a cut-away exploded view of a distal end of one embodiment of the inventive specimen retrieval with the specimen retrieval bag retracted within the housing.

FIG. 4 is a perspective view of a cut-away exploded view of a distal end of one embodiment of the inventive specimen retrieval with the specimen retrieval bag retracted within the housing.

FIG. 5 is a perspective' view of one embodiment of the inventive specimen retrieval with the specimen retrieval bag deployed.

FIG. 6 is a perspective view of a cut-away view of one embodiment of the inventive specimen retrieval from the underside showing the pull ring and not the rod grip, with the specimen retrieval bag deployed.

FIG. 7 is a perspective view of a cutaway exploded view of a deployed specimen retrieval bag in one embodiment of the inventive specimen retrieval with the tip in an active position.

FIG. 8 is a perspective view of a cut-away exploded view of a deployed specimen retrieval bag in another embodiment of the inventive specimen retrieval with the tip in a resting position.

### DETAILED DESCRIPTION OF THE INVENTION

Reference will now be made in detail to the present invention, examples of which are illustrated in the accompanying drawings.

In accordance with one aspect of the invention, a surgical instrument assembly is provided having the capacity to be self-inserted within the body, deploy a specimen retrieval bag and retrieve the specimen. In accordance with one aspect, the inventive assembly has a distal end which is capable of being self-inserted without the need for a trocar. The assembly includes a specimen retrieval bag within a housing. Once the assembly is inserted into the patient and the target tissue, or organ to be removed from the body cavity is in a position relative to the still retracted bag, the specimen retrieval bag is deployed out of the housing. The deployed specimen retrieval bag is moved to encompass the specimen of tissue or an organ, or, the target tissue or organ placed within the deployed and opened bag, and then the specimen retrieval bag is detached, cinched, and separated from the specimen removal device. The specimen retrieval device is removed and the specimen retrieval bag including, the specimen is removed through the initial incision or through a larger incision elsewhere in the body. The device may include a loop grip mechanism, thumb grip mechanism, pistol grip mechanism or other mechanism for the surgeon to deploy and detach the specimen retrieval bag.

It should be noted that although the devices of the present invention are advantageous for various laparoscopic surgeries, including cholecystectomy procedures, they can advantageously be applied to retrieve other organs, such as the uterus or fibroids, for example, and to retrieve tissue such as tumors.

A device or assembly in accordance with the invention advantageously helps by reducing the need for a series of trocars used during surgery. Notably, the need for a larger trocar, such as a 10 mm, 15 mm or 20 mm trocar for insertion of all instruments during surgery, is reduced as typically a conventional known specimen retrieval device requires a larger trocar. The inventive device is self-inserting so no trocar is needed. Further, advantageously, the small diameter size of the inventive device minimizes or eliminates scarring, complications and pain to the patient.

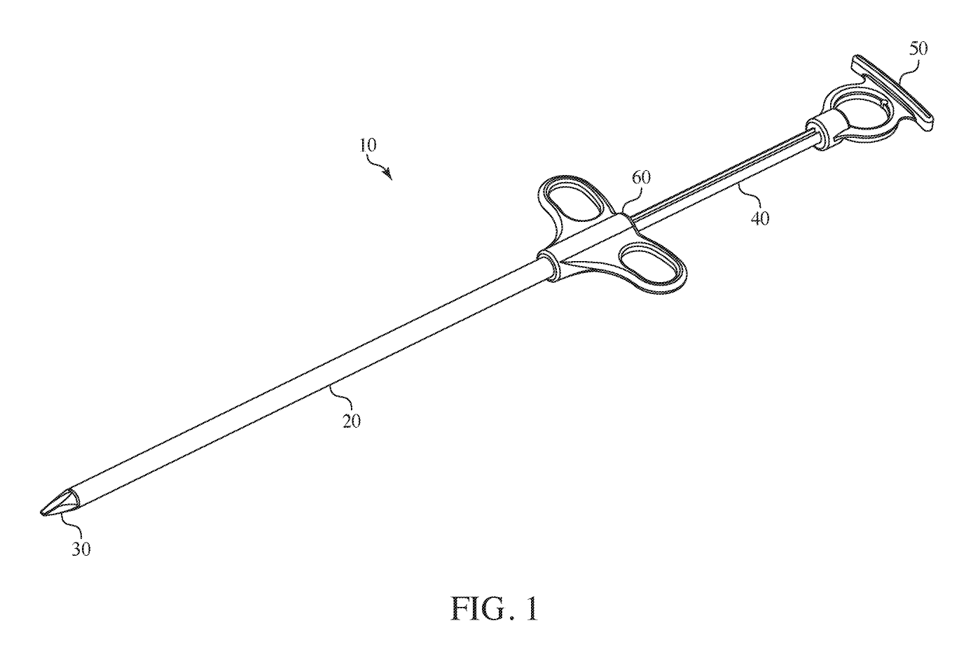

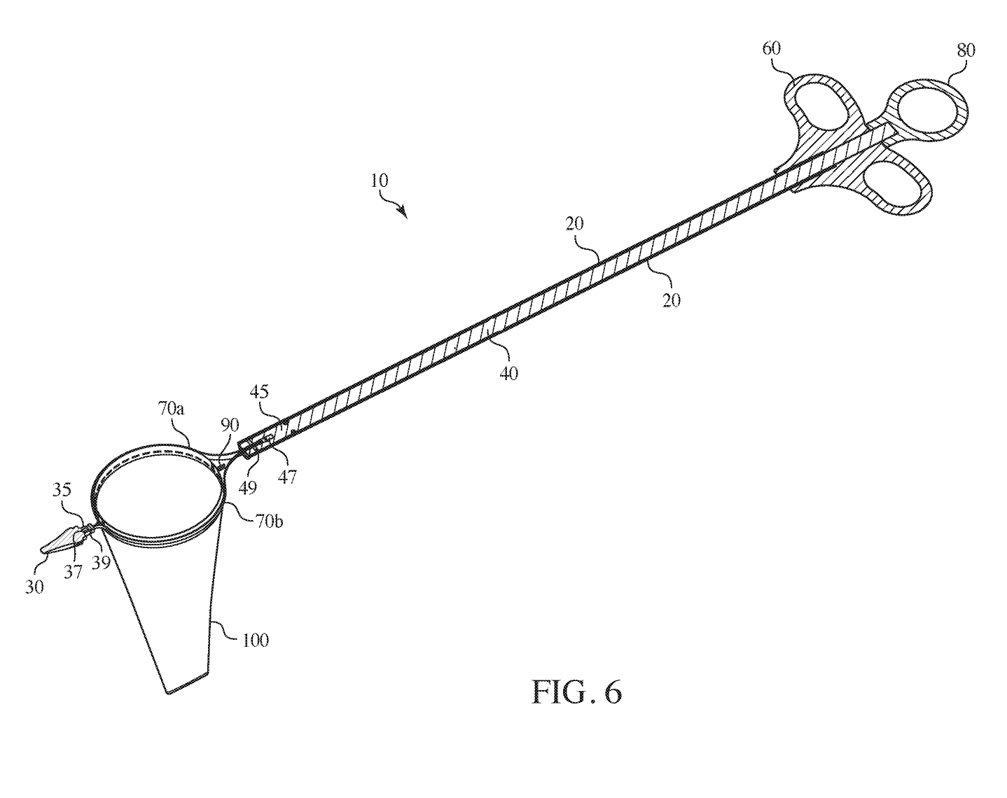

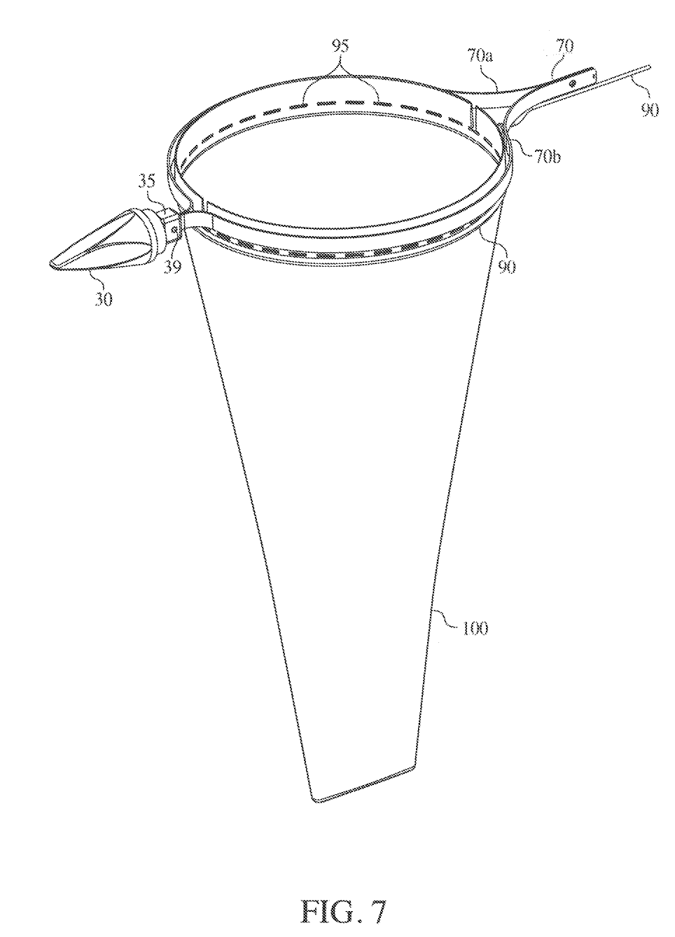

For the purposes of explanation and illustration and not limitation, in accordance with the invention, an exemplary embodiment of a specimen retrieval assembly in an unactuated position is illustrated in FIGS. 1 through 4 and in an actuated position is illustrated in FIGS. 6 through 8. In accordance with these examples, the specimen retrieval device 10 includes a housing 20 having a diameter in a range of about 1 mm to about 50 mm, with a length of about 200 mm in a range of about 50 mm to about 400 mm of length. The housing 20 may be a tube or cannula. The housing 20 may be hollow in which to hold the folded specimen bag (not shown in FIG. 1, 100) and a deployment mechanism for the bag. In one embodiment the diameter of the housing 20 is about 7.5 mm, with other embodiments including but not limited to a housing 20 diameter of about 3 mm, 5 mm, 10 mm, 12.5 mm, 15 mm, 16 mm, 20 mm, 25 mm, 30 mm or larger. The diameter of the housing 20 may be related to the diameter of the open bag (100) as related to the size of the target tissue or organ. For instance, a pediatric patient may require a smaller diameter such as a 3 mm or 5 mm for the diameter of the housing 20.

Housing 20 may be comprised of any material compatible to the human body as the distal end of the housing 20 will be placed within the body cavity for deployment of the bag 100. Such materials include plastics or metals. If a metal, the housing 20 may be comprised of stainless steel and may further be coated with a shrink wrap plastic such as shrinkable polyethylene fiberglass, or polyvinyl chloride of a grade suitable for use in surgical procedures. The housing 20 may be comprised of many known polymers such as polycarbonates or ABS. For cost effectiveness, the housing 20 may be comprised of any plastic capable of sterilization according to regulatory agencies for medical devices.

The dimensions of housing 20 may be a diameter in the range of about 1 mm to about 60 mm, preferably between about 3 mm to about 20 mm. The dimensions of the housing 20 may also be such that the diameter is slightly larger than the maximum diameter of the open specimen retrieval bag 100, either empty or filled and preferably when filled with the target specimen, such as for example without limitation, if the open diameter of the specimen retrieval bag 100 is 5 mm then the diameter of the housing would be between about 7 mm to about 8 mm.

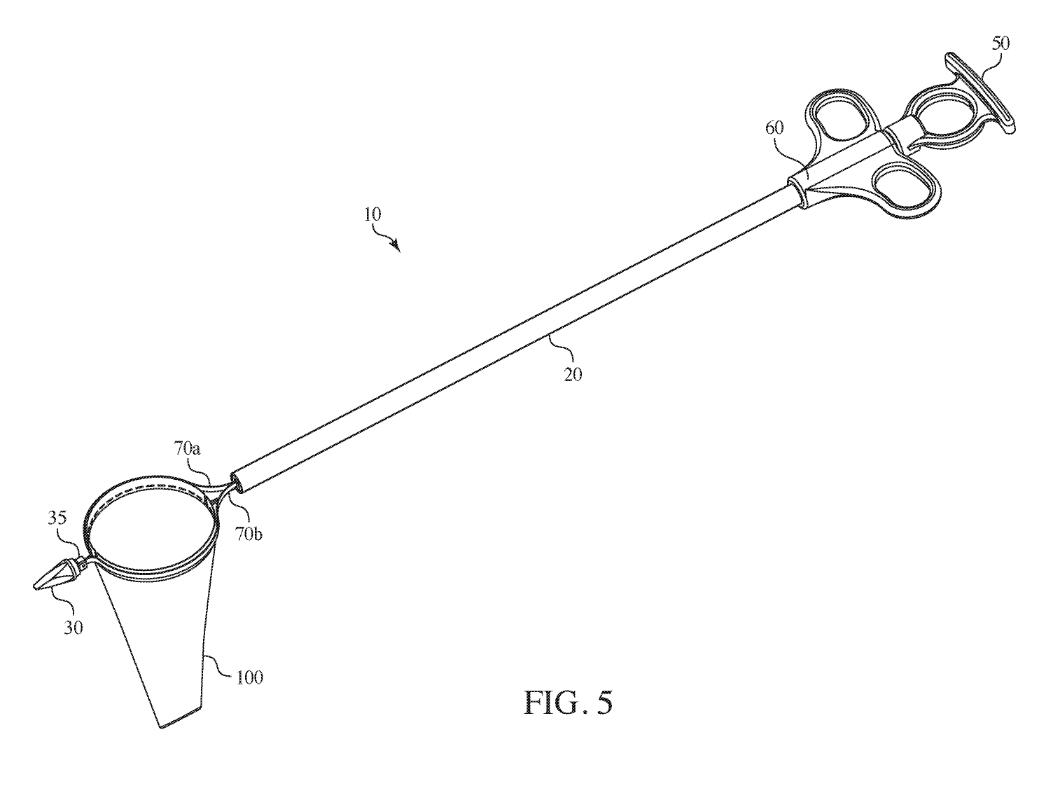

The housing 10 may be hollow and thus includes a channel which houses the folded non-deployed specimen retrieval bag 100 which bag 100 is connected to the deployment mechanism. The deployment mechanism may be a rod 40 and a support means 70. In an unactuated position, the support means 70 is housed within the housing as it is connected to the specimen retrieval bag 100 such that it supports the mouth of the bag 100 when the bag 100 is deployed and open. The rod 40 is partially housed'within the housing 20 at the distal end of the rod 40 and proximal end of the housing 20 in the inventive device's 10 unactuated position while the proximal, end of the rod 40 extends beyond and out of the proximal end of the housing 20 in the unactuated position. To deploy the specimen retrieval bag 100 the rod 40 is pushed in a downward direction toward the self-inserting distal end tip 30 such that the rod 40 resides fully within the housing 20 with just a portion of the proximal end of the rod 40 extending out of the housing 20. In the actuated position the specimen retrieval bag 100 is moved from a stowed position within the distal end of the housing 20 to a deployed position by sliding the distal end 45 of the rod 40 from a retracted position within the housing 20 to an extended position at least partially projecting out of the housing 20. The rod 40 may be connected to a rod grip 50 which the surgeon may use in actuating the inventive device 10, such as a ring grip, finger grip, thumb grip, pistol grip, or other known grips. In FIGS. 1-6, a ring grip is shown as one embodiment of the rod grip 50 which includes a finger portion such that in use the surgeon can place his or her thumb within the diameter of the ring and two fingers on the lower portion of the flat finger rest or grip portion. A further grip is also shown which assists the surgeon in use on that the grip is connected to the outer diameter of the housing 20, on a proximal end of the housing 20, such that the surgeon can grip the housing grip 60 with one hand with gripping the rod grip 50 with the other hand. In use the surgeon would use the housing grip 60, which in this embodiment has two rings oriented across from one another, to stabilize the housing 20 when using force in a downward direction to deploy the folded and unacted specimen retrieval bag 100 via gripping the rod grip 50.

Each of the grips (50, 60) may be comprised of any material, preferably compatible to the human body though neither will reside within the body cavity. Preferred materials are plastics or metals. If a metal, either grip (50, 60) may be comprised of stainless steel and may further be coated with a shrink wrap plastic such as shrinkable polyethylene fiberglass, or polyvinyl chloride of a grade suitable for use in surgical procedures. Either or both of the grips (50, 60) may be comprised of many known polymers such as polycarbonates or ABS. For cost effectiveness, the grips (50, 60) may be comprised of any plastic capable of sterilization according to regulatory agencies for medical devices.

The deployment mechanism includes a rod 40 and further includes a support means 70 connected to the specimen retrieval bag 100. The support means may be a spring, a circular metal or polymer piece, a non-circular metal or polymer piece, one arm, two arms, or other known means to support the mouth of a specimen retrieval bag 100 when open. In one embodiment of the invention the support means is two arms 70a, 10b, which when deployed in an actuated or open position form a circle to support the mouth of a specimen retrieval bag 100 when open. The arms 70a, 70b comprising the support means 70 may be comprised of any flexible materials which is capable of compression when housed within the housing 20 and capable of forming an open position for the mouth of a specimen retrieval bag 100 when open, once deployed and actuated outside of the distal end of the housing 20. The support means 70 may be a spring resilient material to give it a tendency to form a circle, oval or other substantially round shape in a fully deployed position. When retracting, the circle or oval collapses under manual force against spring bias to fit within the housing 20. For instance, the arms 70a, 70b may be comprised of a flexible metal such as stainless steel, may be a high strength stainless steel alloy, titanium, nitinol, steel spring alloys, metal alloys, plastics, combinations thereof. In general, the support means 70 (such as arms 70a, 70b) may be comprised of any material that can be stored in a deformed shape, is resilient, and can be returned via compression or other force to an initial or near initial shape.

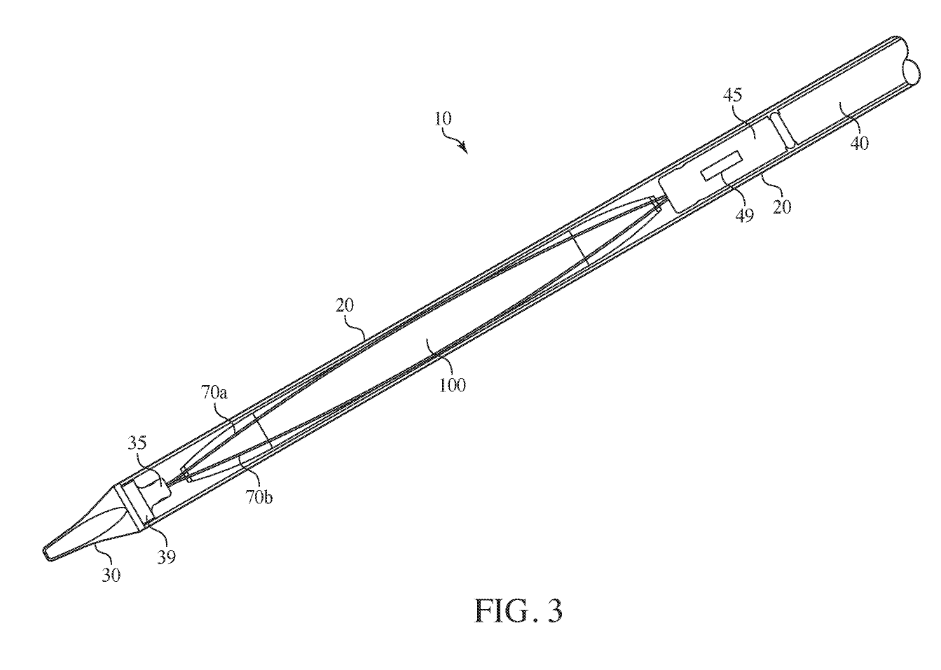

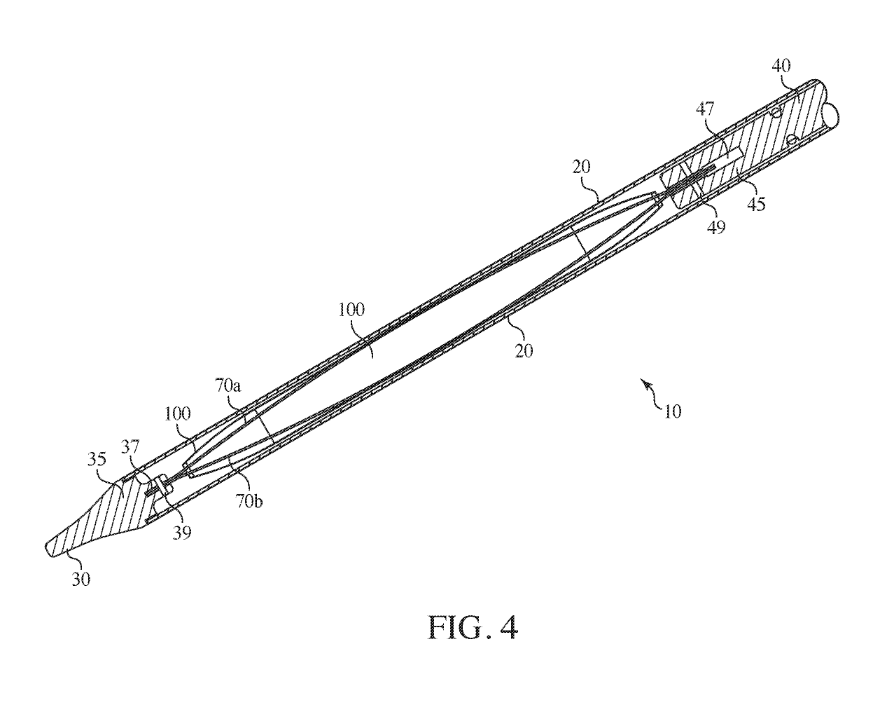

As shown in detail in FIGS. 3 and 4 in one embodiment of the present invention, each of the arms 70a, 70b is connected on its proximal end to the distal end 45 of the rod 40. The distal end 45 of the rod 40 includes an aperture 47 in which the proximal end of each of the arms 70a, 70b may be placed and secured by any known connecting means, such as via a faster, hinge, pin, welding, soldering, crimping, adhesives or another suitable technique, as desired. In one embodiment of the inventive device 10, the proximal end of each of the arms 70a, 70b is placed within aperture 47 and a pin 49 secures that portion of the arms 70a, 70b to the distal end 45 of the rod 40.

The specimen retrieval bag 100 is detachably connected to the support means 70. Typically, a specimen retrieval bag 100 has an unsealed mouth portion located at the top end of the specimen retrieval bag 100 and a sealed portion located at the bottom end of the specimen retrieval bag 100. The specimen retrieval bag 100 may be capable of cinching or closing via a cinching means 90 such as a cinch cord or a string connected to the outer diameter of the specimen retrieval bag 100 at a position below the mouth of the specimen retrieval bag 100. In some specimen retrieval bags 100, the string 90 is connected to the specimen retrieval bag 100 via a channel located on the outer circumference of the specimen retrieval bag 100. Further, the specimen retrieval bag 100 may be capable of detachment from the support means 70 via, for instance, perforations 95 located near mouth and the top of the specimen retrieval bag 100. Other means to detach the specimen retrieval bag 100 may be employed. Further, other means for cinching, closing or sealing the specimen retrieval bag 100 once the specimen is within it may be employed.

The specimen retrieval bag 100 is not, shown in FIG. 1 as it is folded within a channel in the housing 20. FIGS. 1-3 show the specimen retrieval device in an un-actuated configuration but since they are cut-away figures the folded and non-deployed specimen retrieval bag 100 is shown. The specimen retrieval bag 100 may be comprised of elastomeric or polymeric material such as but not limited to polyurethane, polyethylene, polypropylene, vinyl, latex, polymers, plastics, or combinations thereof. Any material which is compatible with the body may be used for the specimen retrieval bag 100. Further, specimen retrieval bag 100 is comprised of a material which is preferably transparent or semi-transparent so that the surgeon can observe the target specimen of tissue or an organ received. In the specimen retrieval bag 100 through an endoscope with naked eyes. The specimen retrieval bag 100 may be of a dimension suitable for encompassing the target specimen, such as for is instance, having a diameter when open from about 2 mm to about 50 mm.

The specimen retrieval bag 100 in one embodiment of the present invention includes an unsealed mouth and a sealed bottom and may be tapered inward, tapered outward or of a square, rectangle, triangle, oval or circular shape overall. The shape of the specimen retrieval bag 100 may be dependent on the target specimen. In another embodiment of the invention, the specimen retrieval bag 100 may not be capable of cinching and instead remains open throughout the procedure and only partially closes upon force by the body wall when the filled specimen retrieval bag 100 is being pulled out of the body cavity and through the body wall and fascia. In another embodiment of the present invention, the specimen retrieval bag 100, may not be capable of cinching but may instead be capable of sealing the open mouth after the specimen is placed within the open and deployed specimen retrieval bag 100.

The specimen retrieval bag 100 has a closed lower end and an open upper end forming a mouth. The mouth is detachably connected to the distal ends of the rod such as by a series of perforations 95. Just below the perforations 95 the specimen retrieval bag 100 includes a cinching or sealing means such as a string 90 which string 90 is sealed within a pouch or channel or sleeve running along the upper end of the specimen retrieval bag 100 configured to cinch or seal the specimen retrieval bag 100 upon detachment. The string 90 is connected at one end to a pull ring 80 (shown in FIG. 6) and the string 90 runs the length of the housing 20 through a channel within the rod 40. Other known detachment means are available for the specimen retrieval bag 100 in addition to perforations, such as scoring or thinning a portion of the material such that it is prone to tear when the string is pulled and the upper end of the specimen retrieval bag 100 is cinched, as well as other means known in the art The string 90 may also be comprised of silk, cotton or nylon or the like. The string 90 may be replaced by other known means to cinch the specimen retrieval bag 100 such as, but not limited to, cords, nylons, plastics, and the like.

The inventive specimen retrieve device 10 further includes a self-inserting distal end tip 30. The distal end tip 30 is which tip is configured to be self-inserting and generally has a non-blunt shape. The distal end tip 30 in FIGS. 1-8 is shown as one embodiment in the shape of a cone. In other embodiments of the present invention the distal end tip 30 may be curved, hooked, pointed, bull-nosed, combinations thereof, or any other non-blunt, shape which has some sharpened form to be self-inserting within skin during use. The distal end tip should be configured such that it is capable of forming an incision on the fascia of a patient, preferably a human being, with the application of force. In one embodiment the distal end tip 30 may be sharp enough that no incision is needed and it punctures the fascia of the patient itself, such as for instance, without limitation, a needle shape. In other embodiments the distal end tip 30 may be inserted within a defect in the skin surface, such as, a prior incision which may or may not be of a smaller diameter than the diameter of the housing 20. In all instances, the distal end tip 30 is self-inserting in that it does not require a port or trocar. However, the use of a trocar or port is not excluded.

In use, the specimen, retrieval device 10 is positioned outside of the body directly over or in the vicinity of the tissue or organ to be retrieved. Force is applied such that the distal end tip 30 penetrates the fascia of the patient. No trocar or port access is needed to insert the specimen retrieval device 10. In some instances the specimen retrieval device 10 may be inserted into a defect or prior incision area In other instances a trocar may be removed, for instance having a diameter or 5 mm, and the inventive specimen retrieval device 10 inserted which has an outer diameter of 7.5 mm so that the specimen retrieval device 10 is self-inserting without need for a trocar. Other diameters of the specimen retrieval device 10 may be employed with other defect widths or other trocar diameter sizes. Once within the body at the target location the specimen retrieval bag 100 is deployed such that it unfolds and the mouth opens via the supporting means 70.

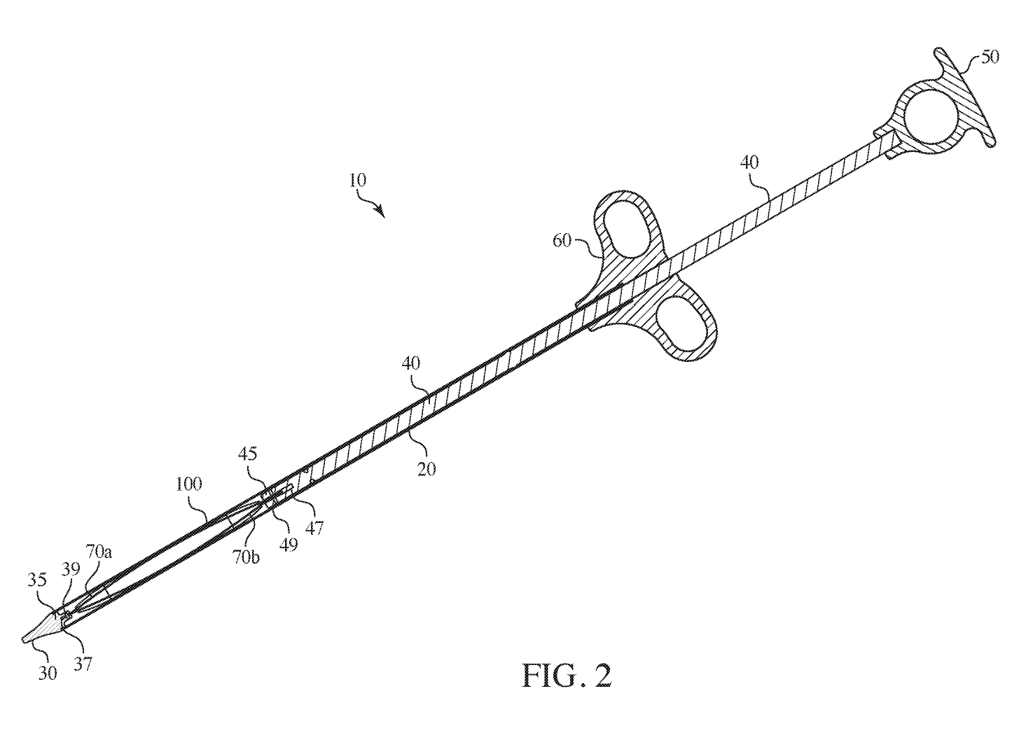

The distal end tip 30 is connected to the support means via any known connecting means. In one embodiment of the present invention, as shown in FIGS. 2-7, the distal end tip 30 is connected to the distal end of each of the arms 70a, 70b. The distal end tip 30 includes an aperture 37 within the proximal end 35 of the distal end tip 30. The distal end of each of the arms 70a, 70b may be placed and secured to the proximal end 35 of the distal end tip 30 by any known connecting means, such as via a faster, hinge, pin, welding, soldering, crimping, adhesives or another suitable technique, as desired. In one embodiment of the inventive device 10, the proximal end of each of the arms 70a, 70b is placed within aperture 37 and a pin 39 secures that portion of the arms 70a, 70b to the proximal end 35 of the distal end tip 30.

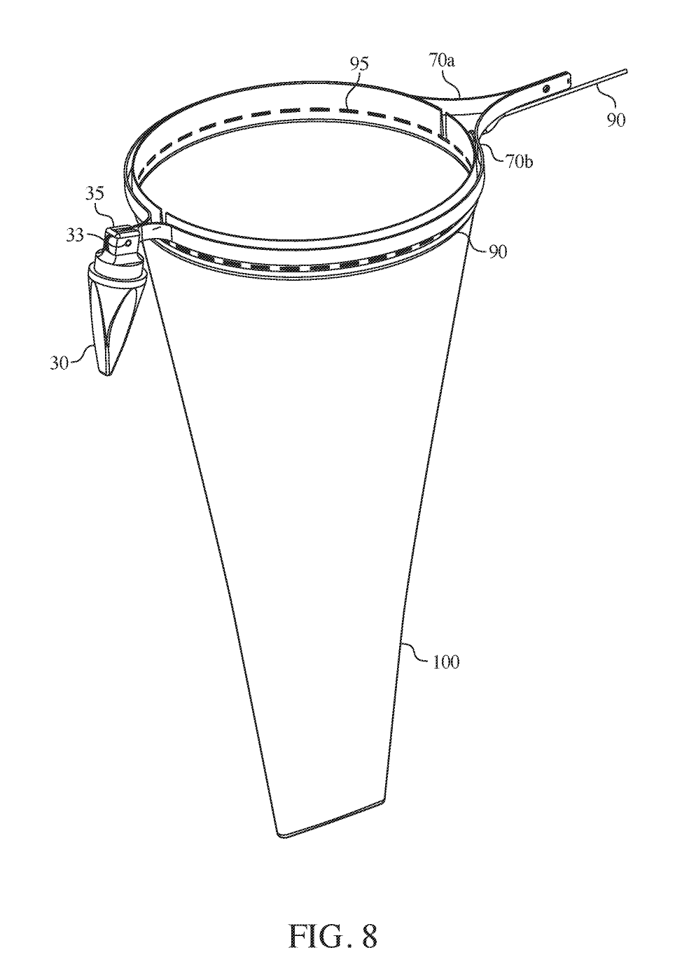

In a further embodiment of the present invention, the distal end tip 30 is connected to the support means via a hinge such the distal end tip 30 may move in a downward direction once within the body cavity. This embodiment may be employed within a body cavity where space is tight so as to reduce any unintentional nicks of non-target tissue or organs by the distal end tip 30. For instance, the specimen retrieval device 10 may be employed in a pediatric patient or in a body location of an adult patient which even when insuffiated, has many non-target tissue and organs within close proximity to the distal end tip 30 when the device 10 is placed within the body cavity. In that instance, the distal end tip 30 may be moved in a downward position once it has been inserted within the body wall and either prior to or after deployment of the specimen retrieval bag. In such an embodiment of the present invention, as shown in FIG. 8, the distal end tip 30 is connected to the distal end of each of the arms 70a, 70b via a hinge 33 such that the distal end tip 30 is movable.

A method of use of the inventive specimen retrieval device 10 includes the initial step of inserting, through sufficient force exerted by the surgeon, the distal end tip 30 into the fascia of a patient, through the body wall and into the body cavity. Such insertion is visualized from within the insufflated body cavity via a camera endoscope already placed within the body cavity. No trocar or access port is needed for insertion of the specimen retrieval device 10. Indeed a trocar may actually be removed and the specimen retrieval device 10 is self-inserted within the existing defect or incision point. The specimen retrieval device 10 is in a non-actuated state at this point in the method.

Once the target tissue or organ is identified within the body cavity, the surgeon will place the distal end of the housing 20 in the vicinity therein and deploy the specimen retrieval bag 100. Deployment occurs when the rod 40 is advanced longitudinally distally through the housing 20 by the operator's force, such as pushing, of the rod grip 50, while maintaining the distal end of the housing 20 within the body cavity by gripping the housing grip 60 to stabilize the housing's 20 location. This movement of the rod 40 pushes the support means 70 and the specimen retrieval bag 100 beyond the distal end arid out of housing 20 and, therefore, the support means 70 resiliently pops open to its substantially round configuration to thereby open the mouth of the specimen retrieval bag 100.

The specimen retrieval device 10 is thus actuated and the specimen retrieval bag 100 unfolds as the specimen retrieval bag 100 is moved from a stowed position to a deployed position by sliding the rod 40 from a retracted position, partially within the housing on its distal end and partially outside the housing at the proximal end of the rod 40, to an extended position of the distal end of the rod 40 at least partially projecting out of the housing 20. Further manipulation of the deployed specimen retrieval bag 100 may occur via surgical instruments such as graspers inserted through the same or a separate incision, cannula or trocar.

The specimen retrieval bag 100 is moved so as to encompass the target tissue or organ within the specimen retrieval bag 100 or the target tissue or organ is placed via graspers or other surgical instruments, within the open and deployed specimen retrieval bag 100. At this point the filled specimen retrieval bag 100 is detached from the main body of the specimen retrieval device 10 and cinched. A pull ring 80, which is connected to the string 90 of the specimen retrieval bag 100, may be grasped and pulled thereby causing the specimen retrieval bag 100 to cinch via the proximal movement of the string and in turn detach from the support means 70 due to the movement of the string and force on the perforations 95 thereby detaching while cinching. At this point the specimen retrieval bag 100, including the target specimen, has a closed mouth and is detached from the support, means 70 but still connected to the device 10 solely via the string 90.

In one embodiment of the inventive specimen retrieval device 10, the string 90 proximately located and connected to the pull ring 80 is severed via a razor (not shown) connected and located within an aperture on a lower portion of the rod grip 50. The string 90 is inserted into the aperture and cut by the razor to allow for complete detachment of the specimen retrieval bag 100 from the specimen retrieval device 10. The fully detached specimen retrieval bag 100 is thus capable of removal separate from the specimen retrieval device 10. Other known detachment means are available such as scissors, knives, razors, non-blunt surfaces and may be used to sever the string (or any other cinching or sealing means).

After the specimen retrieval bag 100 has been detached from the support means 70, the support means 70 is retracted, or pulled, so as to compress and withdraw back into housing 20, whereupon support means 70 refolds back into its pre-deployed relatively straight configuration to permit removal of the specimen retrieval device 10 from the patient's body. The detached string 90 remains partially outside of the patient's body upon removal of the specimen retrieval device 10. The string 80 is used to remove the specimen retrieval bag 100 via force applied, such as pulling, and thus the specimen retrieval bag 100 including the target specimen is removed via the incision formed by the distal end tip 30. In another embodiment of the inventive method, the portion of the string located outside the patient's body may not be used to is remove the specimen retrieval bag 100 and instead the portion of the string 90 located within the patient's body may be used to remove the specimen retrieval bag 40 via a trocar or other incision located on the patient's body such as through the umbilicus. In such an embodiment the portion of the string 90 located within the patient's body is manipulated via another surgical instrument such as a grasper or vacuum instrument.

Another aspect of the present invention is a surgical kit which includes the specimen retrieval device 10. The kit is stored in a sterile sealed package. The kit may include a trocar, a scissors device, a grasper device, and the specimen retrieval device 10. The kit may optionally include a cauterizing device such as a bipolar device. Other optional devices may be included. In one embodiment of the inventive kit all components are single use only and disposable.

Advantages of the inventive specimen retrieval device 10 include the configuration such that it is self-inserting, which reduces the need for a trocar.

Accordingly the incision location is smaller and may cause less surgical damage to the fascia, reduce the total operation time required for the procedure and reduce possible complications. Further, the surgical process for removing a specimen can be simplified by using the inventive specimen retrieval device 10 and the time and cost for the surgery can be reduced.

Thus, the specimen retrieval device 10 may reduce complication surgical processes, time and cost.

Many possible combinations could be within the specimen retrieval device, the methods of use, and the kit or system of the present invention.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

The invention has been described in terms of embodiments thereof, but is more broadly applicable as will be understood by those skilled in the art. The scope of the invention is only limited by the following claims.

We claim:

1. A surgical specimen retrieval assembly, comprising:
a hollow housing;
a deployment rod that has a proximal end out of the housing and a distal end within the housing, which distal end slides from a retracted position within the hollow of the hollow housing to an extended position to at least partially project out of the hollow housing;
a flexible support ring having two portions movable between a folded position that fits within the hollow of the housing and an unfolded position clear of the hollow of the housing, the flexible support ring being connected to the deployment rod so as to move in unison with movement of the deployment rod from the retracted position to the extended position, the two portions of the flexible support, ring being movable from the folded position, to the unfolded position under spring bias as the flexible support ring clears the hollow of the housing and the distal end of the deployment rod enters the extended position from the retracted position;
a specimen bag removably connected to the flexible support ring and movable from a stowed position within the hollow of the hollow housing to a deployed position fully clear, of the hollow housing as the distal end of the deployment and moves from the retracted position into the extended position said specimen bag having a mouth, the mouth being configured to be selectively opened and closed; and
a self-inserting distal end tip connected to a distal end of the flexible support ring, said distal end tip having a region that is shaped to sufficiently insert into skin.

2. The surgical specimen retrieval assembly of claim 1, wherein the self-inserting distal end tip is shaped in a form selected from the group consisting of a cone, bull-nose, curved, hooked, triangle and combinations therein.

3. The surgical specimen retrieval assembly of claim 1, further comprising cinching means connected to the specimen bag for closing the mouth of the specimen bag, the cinching means including a drawstring encircling the specimen bag through a channel on an outer layer of the specimen bag and pulling means for pulling the drawstring, the pulling means being housed within a finger and thumb grip connected to the distal end of the deployment rod.

4. The surgical specimen retrieval assembly of claim 3, wherein said pulling means includes a ring.

5. The surgical specimen retrieval assembly of claim 1, wherein said housing includes a finger grip on an outer diameter of the housing wherein the finger grip includes a first finger ring and a second finger ring each ring oriented across from each other.

6. The surgical specimen retrieval assembly of claim wherein the specimen bag is transparent.

7. The surgical specimen retrieval assembly of claim 1, wherein the specimen bag is translucent.

8. The surgical specimen retrieval assembly of claim 1, wherein the specimen bag has perforations that sever in response to sufficient manual force to detach the specimen bag from the flexible support ring.

9. A method of surgical specimen retrieval, comprising the steps of:
positioning at a location on human skin a sharp means having a region that is shaped to sufficiently be inserted within human skin;
sliding a deployment rod that has a proximal end out of the housing and a distal end within the housing from a retraced position within a hollow of a hollow housing to an extended position to at least partially project out of the hollow housing and moving the self-inserting distal end tip to that is inserts within human skin;
moving at least one of two portions of a flexible support ring between a folded position that fits within the hollow of the housing and an unfolded position clear of the hollow of the housing, moving the flexible support ring, which is connected to the deployment rod, in unison with movement of the deployment rod from the retracted position to the extended position, moving the two portions of the flexible support ring from the folded position to the unfolded position under spring bias as the flexible support ring clears the hollow of the housing and the deployment rod enters the extended position from the retracted position; and
moving a specimen bag, which is removably connected to the flexible support ring, from a stowed position within the hollow of the hollow housing to a deployed position fully clear of the hollow housing as the deployment rod moves from the retracted position into the, extended position so as to thereby open a mouth of the specimen bag in the deployed position.

10. The method of claim 9, further comprising providing the sharp means with a shape in a form selected from the group consisting of, a cone, bull-nose, hook, curve, triangle and combinations therein.

11. The method of claim 9, further comprising closing the mouth of the specimen bag with cinching means that is connected to the specimen bag, the cinching means including a drawstring encircling the specimen bag through a channel on an outer layer of the specimen bag, and pulling the drawstring with pulling means housed within a finger and thumb grip connected to a distal end of the deployment rod.

12. The method of claim 11 herein said pulling is carried out by pulling a ring.

13. The method of claim 9, wherein said housing includes a finger grip on an outer diameter of the housing wherein the finger grip includes a first finger ring and a second finger ring each ring oriented across from each other.

14. The method of claim 9, wherein the specimen bag is transparent.

15. The method of claim 9, wherein the specimen bag is translucent.

16. The method of claim 9, further comprising severing perforations on the specimen bag in response to sufficient manual force to detach the specimen bag from the flexible support ring.

17. A surgical specimen retrieval assembly, comprising:
a hollow housing;
a deployment rod that is configured to move between retracted and extended positions;
a flexible support ring having two portions movable between a folded position that fits within the hollow of the housing and an unfolded position clear of the hollow of the housing, the flexible support ring being connected to the deployment rod so as to move in unison with movement of the deployment rod from the retracted position to the extended position, the two portions of the flexible support ring being movable from the folded position to the unfolded position under spring bias as the flexible support ring clears the hollow of the housing and the deployment rod enters the extended position from the retracted position;
a specimen bag removably connected to the flexible support ring and movable from a stowed position within the hollow of the hollow housing to a deployed position fully clear of the hollow housing as the deployment rod moves from the retracted position into the extended position, said specimen bag having a mouth, the mouth being configured to be selectively opened and closed, the specimen bag being elongated to hang from the flexible support ring with the mouth substantially at, the flexible support ring, the specimen bag also having a closed end in a hanging position spaced away from the flexible support ring; and
an end tip connected to the flexible support ring in a hinged manner to pivot between an outward pointing orientation and a transverse pointing orientation, the outward pointing orientation being in a direction that points substantially in line with the direction that the deployment rod travels to move from the retracted position to the extended position, the transverse pointing orientation being in a direction that points substantially in a direction that is the same as a direction that the closed end travels to reach the hanging position away from the flexible support ring, the end tip tapering into a non-blunt end so as to appear to point in a direction away from where the end tip connects in the hinged manner to the flexible support ring.

18. The surgical specimen retrieval assembly of claim 17, wherein the end tip is shaped in a form selected from the group consisting of a cone, bull-nose, curved, hooked, triangle and combinations therein.

19. The surgical specimen retrieval assembly of claim 17, further comprising cinching means connected to the specimen bag for closing the mouth of the specimen bag, the cinching means including a drawstring encircling the specimen bag through a channel on an outer layer of the specimen bag and pulling means for pulling the drawstring, the pulling means being housed within a finger and thumb grip connected to the distal, end of the deployment rod.

20. The surgical specimen retrieval assembly of claim 17, wherein said housing includes a finger grip on an outer diameter of the housing wherein the finger grip includes a first finger ring and a second finger ring each ring oriented across from each other.
